SEQUENCE LISTING (34 sequences)

SEQ ID NO: 1 - DNA, 23 bp, Homo sapiens
gactacagcaaagatgcccagtg

SEQ ID NO: 2 - DNA, 20 bp, Homo sapiens
gggatacttgggtggaagta

SEQ ID NO: 3 - DNA, 772 bp, Homo sapiens
aaccagattagtagttttttcattcatttggccgtctcagtaagtcaaatattgatactttctactaagtcatcttgccaacacccattttgttatacttatgctgaatctgtttgtcatctcttaagtaagaaaattattgattattttgtggggatttaatttaaaaaaaatggtaatggatactgtaaaggagcattatttggatggtttaaaaacatcttccttgatgggaaaatcttttaaaaggctttctaacttggtgtaattacttgaattaaggaagtgcaatgccattctactgacttagaacaacttttttgacttcctgcaaagaggacccttacagtatttttggagaagttagtaaaaccgaatctgacatcatcacctagcagttcatgcagctagcaagtggtttgttcttagggtaacagaggaggaaattgttcctcgtctgataagacaacagtggagagtatgcatttatttatttacttttacatttttgattcgtttttacagagaaaaacttctacagagataacaattattttgcttttcagaaggacgcatgcagtttcttagggacacggctgacttccagatatgaccatgtatttgtggcttaaactcttggcatttggctttgcctttctggacacagaagtatttgtgacaggtaagtacaaggatattaatattttttaaattattttttctcttttggaggaatgtttgaaatagacataaaaataatttaaatgtgttg

SEQ ID NO: 4 - DNA, 107 bp, Homo sapiens
atttgaaattttctaagagatttttgtttcttctttgcagggcaaagcccaacaccttcccccactggtaagaattaatatttatatttttactaattttattttct

SEQ ID NO: 5 - DNA, 47 bp, Homo sapiens
accacaggttggcacacaaaagttgttaacttaaatatcagggaatg

SEQ ID NO: 6 - DNA, 47 bp, Homo sapiens
atctcaggtttgcgggtcctttagacttgtgcaaatatgaaaagtac

SEQ ID NO: 7 - DNA, 47 bp, Homo sapiens
acctcaggtctgactatgctgctctagtagtgtcttcagttatagat

SEQ ID NO: 8 - DNA, 155 bp, Homo sapiens
gaattaattagcttttattcttctattcattttcttgcagatgcctaccttaatgcctctgaaacaaccactctgagcccttctggaagcgctgtcatttcaaccacaacaataggtgatattaccctcagtcaggcagccacaccatccccatg

SEQ ID NO: 9 - DNA, 107 bp, Homo sapiens
gcaaaattttaataatttacattttttttctccattacagctactactccatctaagccaacatgtggtaagtttatttacttagaatcagcatacctcactttgga

SEQ ID NO: 10 - DNA, 299 bp, Homo sapiens
attctggaaaaataacactcaatgttctattttcttttagatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggtaaatatcaatttatttcttttaataaatttataaaaac

SEQ ID NO: 11 - DNA, 209 bp, Homo sapiens
tgaaatactaatcaagtttatttctgtatcttcttgtcaggggttgaaaagtttcagttacatgattgtacacaagttgaaaaagcagatactactatttgtttaaaatggaaaaatattgaaacctttacttgtgatacacagaatattacctacagatttcagtgtggtaagaatataacattgaccagagaatttttttttgtggc

SEQ ID NO: 12 - DNA, 214 bp, Homo sapiens
atcgatatattcattcgaaatatttctttatttcaggtaatatgatatttgataataaagaaattaaattagaaaaccttgaacccgaacatgagtataagtgtgactcagaaatactctataataaccacaagtttactaacgcaagtaaaattattaaaacagattttgggagtgagtatgttacttgcatttatatgtaaaattgcttctc

SEQ ID NO: 13 - DNA, 200 bp, Homo sapiens
ttatttttcatattacataacattcttattcttttaacaggtccaggagagcctcagattattttttgtagaagtgaagctgcacatcaaggagtaattacctggaatccccctcaaagatcatttcataattttaccctctgttatataaaagagacaggtaatttgtgtagaatttaatttcatcagaaaagagaaat

SEQ ID NO: 14 - DNA, 239 bp, Homo sapiens
ttttaacgacttactaattttttttcacatttttcctcagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcgtaagttatatgttttaatgcttctttccataaatggtaa

SEQ ID NO: 15 - DNA, 289 bp, Homo sapiens
tattaaattattaataacaaatctttcttcattttgatagctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggtaaaagtatgctctctacattactatagtaccaactaca

SEQ ID NO: 16 - DNA, 141 bp, Homo sapiens
tagcaaactaattattttattttttgttactgaaattcaggcctattttcacaatggagactatcctggagaaccctttattttacatcattcaacatcttgtaagttatcactgggctatttattatatatattaagata

SEQ ID NO: 17 - DNA, 189 bp, Homo sapiens
ttgacaatcgttctctgaatgtattatttttcatttctagataattctaaggcactgatagcatttctggcatttctgattattgtgacatcaatagccctgcttgttgttctctacaaaatctatgatctacataagaaaagatcctggtaagagttgattttaaatttttaaataataatggtatta

SEQ ID NO: 18 - DNA, 115 bp, Homo sapiens
ttcttgagaatatagaaacttatttttcctattttcacagcaatttagatgaacagcaggagcttgttgaaaggggtaagtatgtatatttttgctgatgactattccttcccct

SEQ ID NO: 19 - DNA, 190 bp, Homo sapiens
gacacatgtaactagtattgaatctttaatatgtttccagatgatgaaaaacaactgatgaatgtggagccaatccatgcagatattttgttggaaacttataagaggaagattgctgatgaaggaagactttttctggctgaatttcaggtgtgtgttgcttttgttatatgatgataaattcgacatc

SEQ ID NO: 20 - DNA, 171 bp, Homo sapiens
ctgaatctgctgtgatccaagaaatcgttgtttctttcagagcatcccgcgggtgttcagcaagtttcctataaaggaagctcgaaagccctttaaccagaataaaaaccgttatgttgacattcttccttgtgagtatttattgagtgctgaattcccatatattaggct

SEQ ID NO: 21 - DNA, 157 bp, Homo sapiens
ttcactatttcacttgtttatttttcttttccttaaacagatgattataaccgtgttgaactctctgagataaacggagatgcagggtcaaactacataaatgccagctatattgatgtgagtaaaaatttgcatttttcttatacctacatatttc

SEQ ID NO: 22 - DNA, 117 bp, Homo sapiens
tgttcagcaaatgacatatctctgcatgtgttttcaatagggtttcaaagaacccaggaaatacattgctgcacaaggtaatttctttgataatccaatattctttttgaaaaattt

SEQ ID NO: 23 - DNA, 218 bp, Homo sapiens
gaaaatttttcttatcagcttttatttgtttacctcctaggtcccagggatgaaactgttgatgatttctggaggatgatttgggaacagaaagccacagttattgtcatggtcactcgatgtgaagaaggaaacagggtaagaaccaagaagattcatagtgtgggtcttggggttagtaagaaccaagaagattcatagtgtgggtcttggggtta

SEQ ID NO: 24 - DNA, 206 bp, Homo sapiens
aattaaaaattaaaatacttaataattttttaaaatgtagaacaagtgtgcagaatactggccgtcaatggaagagggcactcgggcttttggagatgttgttgtaaagatcaaccagcacaaaagatgtccagattacatcattcagaaattgaacattgtaaatgtgagtttgctttttacataatttttgttttgatactttt

SEQ ID NO: 25 - DNA, 238 bp, Homo sapiens
taaaaaaatcatctcaaagaaaatattatctcttgactagaaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcaggtaggaaaaacgaacaaaaaaaaaaaacaacaacaaaaaa

SEQ ID NO: 26 - DNA, 216 bp, Homo sapiens
ccatgatcatgcctctgcttttttttgcttggtttgccagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggtatgttctaacctttagtgattattctcactttggtttt

SEQ ID NO: 27 - DNA, 230 bp, Homo sapiens
attatcagttaactatctgtatttgttcttgaaattgtaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccaggtaatgatagtgacaacaataataataattacagataact

SEQ ID NO: 28 - DNA, 171 bp, Homo sapiens
cattttaaaggagtttttctgttttttttttttttttcagagacttccttcatataggagctggaggacacagcacattggaaatcaagaagaaaataaaagtaaaaacaggaattctaatgtcatcccatgtatgtagtttatttttttattttttgtatcagataaagt

SEQ ID NO: 29 - DNA, 214 bp, Homo sapiens
ttttttcaaggaagacttactactgatttatttcacatagatgactataacagagtgccacttaaacatgagctggaaatgagtaaagagagtgagcatgattcagatgaatcctctgatgatgacagtgattcagaggaaccaagcaaatacatcaatgcatcttttataatggtaggtacttaaattgccaaaacccaagatccaaacattt

SEQ ID NO: 30 - DNA, 215 bp, Homo sapiens
tctgtagtaacgaagtcccaccctttttttgtctgaaaagagctactggaaacctgaagtgatgattgctgctcagggaccactgaaggagaccattggtgacttttggcagatgatcttccaaagaaaagtcaaagttattgttatgctgacagaactgaaacatggagaccaggtttgtacttttgaggattttcttttaagcctttctgtca

SEQ ID NO: 31 - DNA, 203 bp, Homo sapiens
aaataggaaacaaattgttgaattgtcttcttttatctaggaaatctgtgctcagtactggggagaaggaaagcaaacatatggagatattgaagttgacctgaaagacacagacaaatcttcaacttatacccttcgtgtctttgaactgagacattccaaggtatggaaacaatttggggagtatatttctttgatataat

SEQ ID NO: 32 - DNA, 259 bp, Homo sapiens
acttcactccagttaatgctctcttcaattctgattttagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcaggtgcgtgggatttggtagaatgtgctctcaaaatcataat

SEQ ID NO: 33 - DNA, 216 bp, Homo sapiens
gagaagtaggattattttctatcttttctttcttttatagggatggatctcagcaaacgggaatattttgtgctttgttaaatctcttagaaagtgcggaaacagaagaggtagtggatatttttcaagtggtaaaagctctacgcaaagctaggccaggcatggtttccacattcgtaagtatccttcaccattgcttttaacatgctcggaatt

SEQ ID NO: 34 - DNA, 1375 bp, Homo sapiens
aactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagccttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatacttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttctccaatgcttagaatgtttttaccaggaatggatgtcgct

(19) United States
(12) Patent Application Publication
(10) Pub. No.: US 2003/0008276 A1
(43) Pub. Date: Jan. 9, 2003

(54) Screens for susceptibility to immunodeficiency and viral disease

(75) Inventors: Elma Tchilian, Berkshire (GB); Peter Beverley, Berkshire (GB)

Correspondence Address: Elizabeth R. Plumer, Wolf, Greenfield & Sacks, P.C., Federal Reserve Plaza, 600 Atlantic Avenue, Boston, MA, 02210-2211, US

(73) Assignee: The Edward Jenner Institute for Vaccine Research Compton, Berkshire (GB)

(21) Appl. No.: 10/020,758

(22) Filed: Oct. 30, 2001

(30) Foreign Application Priority Data
Jun. 14, 2001 (GB) 0114512.74

(51) Int. Cl. C12Q 1/70; C12Q 1/68

(52) U.S. Cl. 435/5; 435/6

(57) ABSTRACT

Methods of screening human subjects for susceptibility to viral disease and/or a predisposition to developing more severe viral disease and also methods of screening human subjects for susceptibility to developing immunodeficiency and/or a predisposition to developing more severe immunodeficiency.

### RELATED APPLICATIONS

This application claims priority under 35 U.S.C. §119 to GB 0114512.7, filed June 14, 2001, the entire contents of which is hereby incorporated by reference.

### FIELD OF THE INVENTION

The invention relates to associations between genetic variation in the gene encoding CD45 and human disease. In particular the invention provides methods of screening human subjects for susceptibility to viral disease and/or a predisposition to developing more severe viral disease and also methods of screening human subjects for susceptibility to developing immunodeficiency and/or a predisposition to developing more severe immunodeficiency.

### BACKGROUND

The leucocyte common antigen CD45 is an abundant tyrosine phosphatase, expressed on all leucocytes (Trowbridge, I. S., and M. L. Thomas. 1994. Ann. Rev. Immunol. 12:85). The phosphatase activity of CD45 is essential for lymphocyte antigen receptor signal transduction. Both CD45 deficient mice (Kishihara, K. et al., 1993. Cell 74:143; Byth, K. et al. 1996. J. Exp. Med. 183:170) and humans (Kung, C. et al, 2000, Nature Medicine, 6: 343; Tchilian, E. Z. et al., 2001, J. Immunol., 166: 1308) are severely immunodeficient, with very few peripheral T lymphocytes and impaired T and B cell responses.

Multiple CD45 isoforms can be generated by alternative splicing of exons A, B, and C of the extracellular domain (Saga, Y. et al., 1986. Proc Natl Acad Sci USA, 83: 6940; Streuli, M. el al., 1987, J. Exp. Med., 166: 1548). In humans, naive T cells express high molecular weight CD45 isoforms, recognised by CD45RA monoclonal antibodies (mAbs), but activation of the cells results in a change to expression of low molecular weight isoforms, detected by a CD45RO mAb (Akbar, A. N., et al., 1988, J. Immunol., 140: 2171). These two major subsets of T lymphocytes, expressing CD45RA and CD45RO have been termed naive and memory cells.

Genetically determined abnormal CD45 splicing has been described in humans (Schwinzer, R., and K. Wonigeit, 1990, J. Exp. Med. 171:1803.). Activated or memory lymphocytes in these individuals continue to express both high and low molecular weight CD45 isoforms in contrast to the normal pattern of low molecular weight isoform expression. A C to G transversion at position 77 (C77G) in the fourth or A exon of the gene encoding CD45, has been shown to prevent the normal splicing of this exon in the affected individuals (Thude, R. et al., 1995, Eur. J. Immunol., 25: 2101; Zilch, C. F. et al., 1998, Eur. J. lmmunol., 28: 22) by disrupting a strong exonic splicing silencer (Lynch, K. W. a. W., 2001, J. Biol. Chem). Thel C77G polymorphism has been shown to correlate with development of multiple sclerosis in some families (Jacobsen, M. et al., 2000, Nat Genet, 26: 495) suggesting that the regulation of CD45 alternative splicing may be important in the development of some autoimmune disorders.

### SUMMARY OF THE INVENTION

The present inventors have investigated the pattern of CD45 expression in HIV infection and have demonstrated a statistically significant association between the C77G mutation and HIV-1 infection.

Further observations made by the present inventors provide evidence that the C77G mutation may be a marker for general susceptibility to viral infection and/or a marker for disease severity following viral infection. Accordingly, the inventors have developed screens for determining susceptibility of human subjects to viral infection and/or identifying individuals pre-disposed to developing more severe disease following viral infection based on screening for the presence or absence of the C77G mutation at the protein, MYNA or genomic DNA level.

Therefore, in accordance with a first aspect of the invention there is provided a method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

In the context of this application the terms “gene encoding CD45” and “CD45 gene” are used interchangeably and refer to a gene, also referred to as the PTPRC gene located at gene map locus Iq31-32 (OMIM accession 151460). The complete sequence of the gene is available via publicly accessible genome sequence databases. A list of GenBank accession numbers for individual exons of the gene is provided.

The method of the invention preferably comprises screening the genome of the individual for one or more polymorphic variants of the gene encoding CD45 which have previously been demonstrated to show statistically significant association with susceptibility to viral disease and/or severity of viral disease, for example in a population-based genetic association study.

The invention also contemplates screens based on polymorphic variants (whether or not within the CD45 gene) which have not themselves been shown to be associated with susceptibility to viral infection and/or severity of disease in a population-based study but which are either in linkage disequilibrium with or in close physical proximity to a marker in the CD45 gene shown to be associated with susceptibility to viral infection.

As would be readily apparent to persons skilled in the art of human genetics,

“linkage disequilibrium” occurs between a marker polymorphism (e.g. a DNA polymorphism which is “silent”) and a functional polymorphism (i.e. genetic variation which affects phenotype or which contributes to a genetically determined trait) if the marker is situated in close proximity to the functional polymorphism. Due to the close physical proximity, many generations may be required for alleles of the marker polymorphism and the functional polymorphism to be separated by recombination. As a result they will be present together on the same haplotype at higher frequency than expected, even in very distantly related people. As used herein the term “close physical proximity” means that the two markers/alleles in question are close enough for linkage disequilibrium to be likely to arise.

In a preferred embodiment the method of the invention comprises screening for the presence or absence in the human subject of the C77G mutation in the gene encoding CD45, wherein subjects having at least one mutant allele are scored as being susceptible to viral infection.

As will be illustrated in the accompanying Example, a mutation (C to G transversion) in the fourth or “A” exon of the CD45 gene has been shown to be associated with HIV-1 infection. In addition, the C77G mutation has been found in a patient with common variable immunodeficiency with persistent viral infection and prolonged excretion of polio virus (this patient was previously described by Misbah et al., Postgrad Med J, 1991, Vol: 67, 301-303; see Example 3) and in a patient infected with EBV (data not shown). Furthermore, the inventors have shown the C77G mutation to be present in patients diagnosed with haemoplhagocytic lymphohistiocytosis (HLH) (see Example 2). Sporadic cases of HLH are often provoked by viral infection in childhood (Dreyer, et al., Am J Pediatr Hematol Oncol, Vol: 13, 476). In addition work with transgenic mice expressing single CD45 isoforms indicates that those expressing high molecular weight isoforms show defective immune responses (unpublished data). Together these observations suggest that the C77G mutation is predictive of a general susceptibility or pre-disposition to viral infection.

In the context of the invention, the process of screening for the presence or absence of a mutation or allelic variant in the genome of an individual may advantageously comprise screening for the presence or absence in the genome of the subject of both the common or wild type allele and the variant or mutant allele or may comprise screening for the presence or absence of either individual allele, it generally being possible to draw conclusions about the genotype of an individual at a polymorphic locus having two alternative allelic forms just by screening for one or other of the specific alleles.

The step of screening for the presence or absence of a mutation or allelic variant in the genome of a subject, also referred to herein as “genotyping”, can be carried out using any suitable methodology known in the art and it is to be understood that the invention is in no way limited by the precise technique used to perform such genotyping.

Known techniques for the scoring of single nucleotide polymorphisms include mass spectrometry, particularly matrix-assisted laser desorptioon/ion izatioon time-of-flight mass spectrometry (MALDI-TOF-MS), single nucleotide primer extension and DNA chips or microarrays (see review by Schafer, A. J. and Hawkins, J. R. in Nature Biotechnology, Vol 16, pp33-39 (1998)). The use of DNA chips or microarrays could enable simultaneous genotyping at many different polymorphic loci in a single individual or the simultaneous genotyping of a single polymorphic locus in multiple individuals. SNPs may also be scored by DNA sequencing.

In addition to the above, SNPs are commonly scored using PCR-based techniques, such as PCR-SSP using allele-specific primers (described by Bunce M, et al., Tissue Antigens, 1995; 50: 23-31). This method generally involves performing DNA amplification reactions using genomic DNA as the template and two different primer pairs, the first primer pair comprising an allele-specific primer which under appropriate conditions is capable of hybridising selectively to the wild type allele and a non allele-specific primer which binds to a complementary sequence elsewhere within the gene in question, the second primer pair comprising an allele-specific primer which under appropriate conditions is capable of hybridising selectively to the variant allele and the same non allele-specific primer. A still further PCR-based technique for scoring SNPs is PCR ELISA.

If the SNP results in the abolition or creation of a restriction site, as is the case with the C77G mutation in the CD45 gene, genotyping can be carried out by performing PCR using non-allele specific primers spanning the polymorphic site and digesting the resultant PCR product using the appropriate restriction enzyme (also known as PCR-RFLP). Restriction fragment length polymorphisms, including those resulting from the presence of a single nucleotide polymorphism, may be scored by digesting genomic DNA with an appropriate enzyme then performing a Southern blot using a labelled probe corresponding to the polymorphic region (see Molecular Cloning: A Laboratory Manual, Sambrook, Fritsch and Maniatis, Cold Spring Harbor Laboratory, Cold Spring Harbor, N.Y.).

The known techniques for scoring polymorphisms are of general applicability and it will be readily apparent to persons skilled in the art that known techniques may be adapted for the scoring of single nucleotide polymorphisms in the CD45 gene. In the case of the C77G mutation, the preferred technique for scoring this mutation is PCR followed by digestion of the PCR product with the enzyme MspI, as described in the accompanying Example. However, the invention is not intended to be limited to the use of this technique.

Genotyping is preferably carried out in vilro, and is most preferably performed on isolated genomic DNA prepared from a suitable tissue sample obtained from the subject under test. Most commonly, genomic DNA is prepared from a sample of whole blood, according to standard procedures which are well known in the art.

In a further aspect the invention provides a method of screening a human subject for susceptibility to viral infection and/or pre-disposition to developing severe disease following viral infection which comprises evaluating the pattern of CD45 mRNA expression in the subject, wherein the presence of an abnormal pattern of CD45 MYNA expression associated with the presence of a C77G mutant allele of the gene encoding CD45 is taken as an indication that the subject is more susceptible to viral infection and/or more pre-disposed to developing severe disease following viral infection, as compared to subjects who do not carry a C77G mutation.

The term “abnormal pattern of CD45 mRNA expression associated with the presence of a C77G mutant allele of the gene encoding CD45” refers to the variant CD45 splicing phenotype described by Thude et al., Eur J Immunol, 1995, Vol: 25(7), 2101-6 and shown to be associated with heterozygosity for the C77G mutation. Individuals homozygous for the C77G mutation are expected to show an exaggeration of the mRNA expression pattern observed in heterozygotes.

Suitable RNA analysis techniques which may be used to determine the pattern of CD45 mRNA expression in accordance with the invention include, but are not limited to, RT-PCR, starting from a sample of total or mRNA prepared from a tissue which expressed CD45 (e.g. PBLs), Northern blotting and RNAse protection.

In a still further aspect the invention provides a method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression associated with the presence of a C77G mutant allele of the gene encoding CD45 is taken as an indication that the subject is more susceptible to viral infection and/or more pre-disposed to developing severe disease following viral infection, as compared to subjects who do not carry a C77G mutation.

The term Apattern of CD45 protein expression associated with the presence of a C77G mutant allele of the gene encoding CD45″ refers to the variant pattern of expression of CD45 protein isoforms on peripheral T cells shown to be associated with heterozygosity for the C77G mutation, as described by Thude et al., Eur J Immunol, 1995, Vol: 25(7), 2101-6. The normal pattern of CD45 protein expression is characterised by loss of expression of the CD45RA isoform and gain in expression of CD45RO after T cell activation. Individuals heterozygous for C77G are characterised continuous expression of the CD45RA isoform on activated and memory T cells, i.e. the T cells remain CD45RA/RO double positive after activation. Individuals homozygous for the C77G mutation are expected to show very little expression of CD45RO at the cell surface.

Analysis of the CD45 protein isoform expression pattern on peripheral T cells is preferably carried out using flow cytometry, as described in the accompanying example. Individuals heterozygous for C77G are characterised by the absence of a CD45RA negative population of leucocytes. Further suitable techniques which may be used to assess the pattern of expression of CD45 isoforms include immunoprecipitation and Western blotting.

The screening methods of the invention may be used to identify human subjects who are susceptible or pre-disposed to viral infection by virtue of their genetic make-up. This may allow intervention with preventative therapies aimed at boosting immune function. Screening for increased susceptibility to viral infections and/or for risk of developing more severe virus-induced disease would be important for individuals at increased risk of life threatening virus infections. These may include, for example, gay men and intravenous drug users or medical personnel working in renal dialysis units. “At risk” individuals may be counselled or excluded from high risk situations and measures may be taken to ensure that vaccination results in protective antibody titres in these individuals where a vaccine is available. Screening may also be useful for predicting whether individuals with chronic viral infection, such as for example Hepatitis B or C, are likely to be refractory to expensive immunotherapy.

The inventors have also made observations which provide evidence that carriage of the C77G mutation is predictive of susceptibility to immunodeficiency diseases other then severe combined immune deficiency (SCID) and/or a pre-disposition to developing more severe disease. Evidence for an association between C77G and predisposition to developing more severe immunodeficiency disease is provided in particular by the inventors' observation that the C77G mutation is present in a patient with common variable immunodeficiency and prolonged excretion of polio virus.

Therefore, in a still further aspect the invention provides a method of screening a human subject for susceptibility to developing immunodeficiency disease other than severe combined immune deficiency and/or pre-disposition to a developing a more severe form of the immunodeficiency disease, which method comprises screening for the presence or absence in the genome of said subject of one or more polymorphic variants or mutations in the gene encoding CD45 or of one or more polymorphic variants in linkage disequilibrium with or in close physical proximity to a polymorphic locus in the gene encoding CD45.

In a preferred embodiment this method comprises screening for the presence or absence in the human subject of the C77G mutation in the gene encoding CD45, wherein subjects having at least one mutant allele are scored as being more susceptible to developing immunodeficiency and/or more likely to develop a severe form of immunodeficiency than subjects who do not carry a C77G mutation.

The invention further provides a method of screening a human subject for susceptibility to developing immunodeficiency disease other than severe combined immune deficiency and/or pre-disposition to a developing a more severe form of the immunodeficiency disease, which method comprises evaluating the pattern of CD45 mRNA expression in the subject, wherein the presence of an abnormal pattern of CD45 mRNA expression associated with the presence of a C77G mutant allele of the gene encoding CD45 is taken as an indication that the subject is more susceptible to developing immunodeficiency and/or more likely to develop a severe form of immunodeficiency than subjects who do not carry a C77G mutation.

The invention further provides a method of screening a human subject for susceptibility to developing immunodeficiency disease other than severe combined immune deficiency and/or pre-disposition to a developing a more severe form of immunodeficiency disease, which method comprises evaluating the pattern of CD45 protein expression in the subject, wherein the presence of an abnormal pattern of CD45 protein expression associated with the presence of a C77G mutant allele of the gene encoding CD45 is taken as an indication that the subject more susceptible to developing immunodeficiency and/or more likely to develop a severe form of immunodeficiency than subjects who do not carry a C77G mutation.

In the context of this application the term “immunodeficiency” disease other than severe combined “immune deficiency” encompasses, but is not limited to, common variable immunodeficiency, selective IgA and/or IgG deficiency, DiGeorge syndrome, X-linked lymphoproliferative syndrome, Bloom's syndrome and Ataxia telangiectasia.

Tchilian et al. (J. Immunol., 2001, 166: 1308-1313) have previously reported a homozygous 6-bp deletion in the CD45 gene in a patient diagnosed with SCID. However, there have been no published reports of the significance of CD45 mutations in clinically milder immunodeficiencies such as, for example, common variable immunodeficiency, selective IgA and/or IgG deficiency, DiGeorge syndrome, X-linked lymphoproliferative syndrome, Bloom's syndrome, Ataxia telangiectasia. In particular, there has been no previous suggestion that C77G heterozygotes exhibit increased susceptibility to immunodeficiency and/or increased disease severity. By definition SCID is a very severe form of immunodeficiency, helnce the presence of a C77G mutation in addition to the causative SCID abnormality is unlikely to have a significant effect on disease severity.

The above methods of screening for susceptibility to developing immunodeficiency disease and/or pre-disposition to a developing a more severe form of immunodeficiency disease may be carried out using the same methodology as described previously for the screens for determining susceptibility to viral infection and/or pre-disposition to developing severe viral disease.

In a still further aspect the invention provides a method of screening a human subject for susceptibility to haemophagocytic lymphohistiocytosis, which method comprises screening for the presence or absence in the human subject of the C77G mutation in the gene encoding CD45, wherein subjects having at least one mutant allele are scored as being more susceptible to haemophagocytic lymphohistiocytosis, as compared to subjects who do not carry a C77G mutation.

As illustrated in the accompanying Examples, the C77G mutation has been found in patients diagnosed with haemophagocytic lymphohistiocytosis. The C77G mutation may therefore be exploited as a marker for HLH.

C77G may be a factor provoking HLH, possibly in combination with mutations in other genes such as, for example, the perforin gene PFR1. In this connection it should be noted that studies with transgenic mice suggest that expression of a high molecular weight CD45 isoform alone causes immunodeficiency and these mice can not generate any cytotoxic T cell responses or neutralising antibodies after viral infection (Kozieradzki, I., et al. 1997. J. Immullol. 158:3130.). Furthermore recent data shows that CD45 can dephosphorylate the Janus kinases, which down-regulate the cytokine and interferon receptor activation involved in differentiation, proliferation and anti-viral immunity of hemopoietic cells (Irie-Sasaki, J., et al. 2001. Nature 409:349.). Patients who are heterozygotes for C77G would be expected to express a more subtle form of immunodeficiency but this may contribute to precipitating the disease. As aforesaid, sporadic HLH cases are often provoked by viral infection later in childhood (Dreyer, Z. E., et al., 1991. Am. J. Pediatr. Hematol. Oncol. 13:476.), indicating that individuals carrying the C77G mutation may have a higher risk of viral infection. Finally, it is possible that although mutations in CD45 may be associated with HLH, the primary cause of disease may be due to mutations in as yet unidentified genes. However, this does not affect the utility of mutations in CD45, in particular C77G, as markers for susceptibility to HLH.

Screening for the presence of the C77G mutation in HLH patients may also be useful diagnostically, for example as a method of investigating underlying genetic basis for HLH in patients previously diagnosed with HLH or suspected of having the disease.

### BRIEF DESCRIPTION OF THE DRAWINGS

The invention will be further understood with reference to the following, non-limiting, Experimental Example and the accompanying Figures in which:

FIG. 1: (A) Illustrates detection of exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele; (B) shows the results of FACS analysis to investigate the pattern of CD45 expression in human peripheral T cells pre- and post- stimulation. PBMC were stimulated with 1 μg/ml PHA and on days 0 and day 10 stained with isoform-specific CD45RO-PE and CD45RA-FITC antibody conjugates and with a CD3-APC antibody conjugate. Analysis was performed on gated CD3+ cells. Panels A and B show the normal pattern of CD45 expression pre- and post- stimulation: T cell activation is associated with a loss in CD45RA and a gain in expression of CD45RO. Panels C and D show the pattern of CD45 expression pre- and post- stimulation in a C77G heterozygote: the CD45RA population is largely absent and the T cells remain CD45RA/RO double positive after activation.

FIG. 2: shows a family tree indicating the CD45 genotype and phenotype in each member of a family including an individual with HLH (family W). Thie patient with HLH (5) is indicated by an asterisk.

(A) Identification of the CD45 exon A (C77G) mutation in family W. The C77G transversion introduces a new restriction site for MspI, which cleaves the mutant PCR product into two fragments of 72 and 83 bp (lanes 2, 4, 5 and 6). The presence of an undigested band of 155 bp indicates the presence of the wild type allele in the father and older brother (lanes 1 and 3).

(B) Expression of CD45 isoforms in human peripheral T cells. PBMC were stained with isoform specific CD45RA-FITC and CD45RO-PE together with CD3-APC mAbs. Analysis was performed on gated CD3+ cells. The normal pattern of CD45 isoform expression is characterised by the presence of single CD45RA+ and single CD45RO+ cells. Abnormal CD45 expression was seen in the patient (5), his mother (2) and two siblings (4 and 6). The father (1) and brother (3) have normal CD45 pattern of expression.

FIG. 3: illustrates expression of CD45 isoforms in a patient with a common variable immunodeficiency and a history of prolonged faecal excretion of poliovirus.

(A) Detection of exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele.

(B) Flow cytometric analysis of CD45 splicing in CVID patients. Anti-CD3+ lymphocytes stained with CD45RA-FITC and CD45-RO antibodies are shown. Variant CD45 splicing in the patient with prolonged poliovirus excretion can be identified by the absence of the single CD45RO+ population.

### EXAMPLES

### Example 1

### Association Between C77G and HIV Infection

Genomic DNA samples and cryopreserved PBMC were obtained from 182 HIV-1 infected patients enrolled at the St Stephen's Clinic, Chelsea and Westminster Hospital, as a part of a functional immunological study. An additional 15 DNA samples from individuals identified as HIV-1-infected at seroconversion, were supplied by Dr P. Borrow. Ethical approval was obtained and the patients gave written consent. The control group consisted of 236 healthy volunteer blood donors, obtained through the local blood bank of the UK National Blood Transfusion Service.

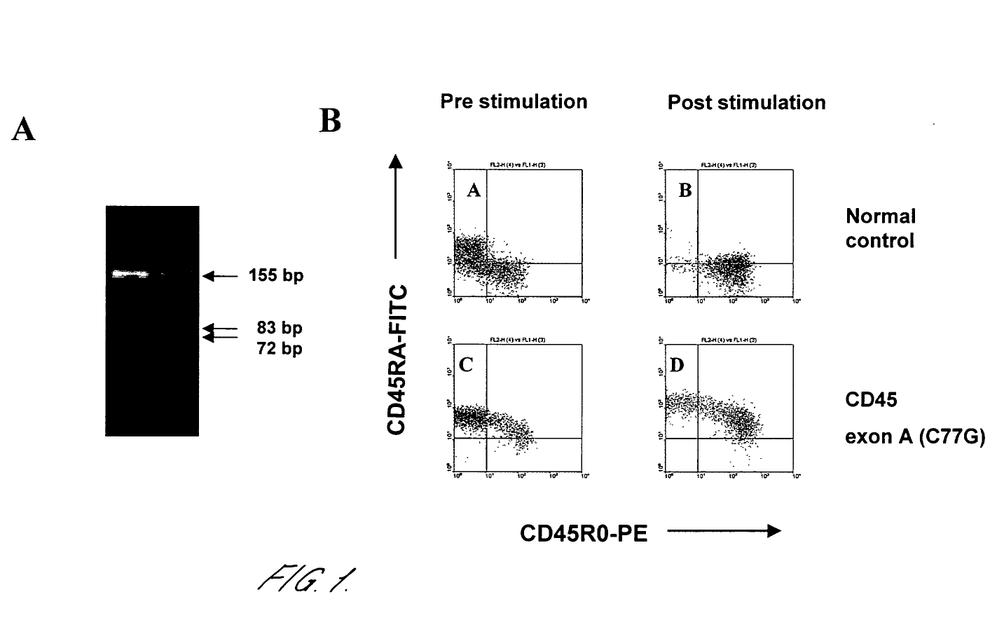

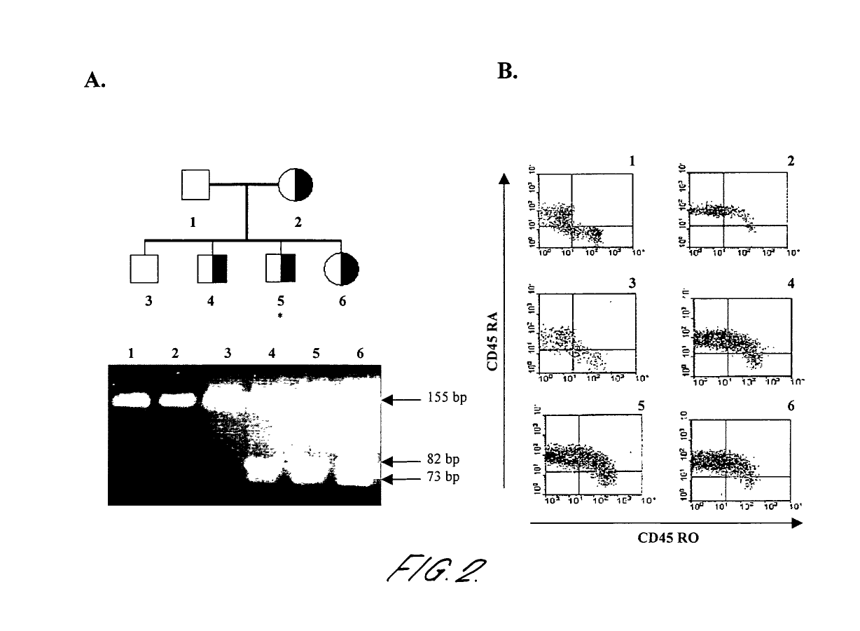

The detection of exon A (C77G) was performed on genomic DNA amplified by PCR using forward (5′-GACTACAGCAAAGATGCCCAGTG-3′, SEQ ID NO: 1) and reverse primers (5′-GGGATACTTGGGTGGAAGTA-3′, SEQ ID NO: 2). The C77G transition introduces a new restriction site for MspI which cleaves the mutant PCR product into two fragments of 72 and 83 bp. The presence of an undigested band of 155 bp indicates the presence of the wild type allele (illustrated in FIG. 1A). The presence of the CD45 exon A mutant allele was confirmed by sequencing and flow cytometric analysis on C77G positive samples. PBMC were stimulated with PHA and on days 0 and day 10 stained with isoform specific CD45RO-PE and CD45RA-FITC antibody conjugates (obtained from Dako and Sigma, respectively) together with CD3-APC antibodies (obtained from Pharmingen). Analysis was performed on gated CD3+T cells. The normal pattern of CD45 splicing is characterised by loss of CD45RA and gain in expression of CD45RO associated with the activated/memory function (A and B, FIG. 1). Variant CD45 splicing can be identified by the absence of the single CD45RO+ population and even after 10 days of stimulation the T cells remain CD45RA/RO double positive (C and D, FIG. 1).

Using PCR and Msp I digestion analysis 11 individuals with the exon A (C77G) mutation were identified out of 197 HIV-1 patients and 4 out of 236 healthy donors (Table 1). The presence of the C77G mutation in these individuals was confirmed by flow cytometric analysis of CD45 protein expression. Using two-tailed Fisher's exact test to test for the association between C77G mutation and HIV-1 infection, a statistically significant association was demonstrated (p=0.03).

The results of this study clearly indicate that exon A (C77G) transversion and abnormal CD45 splicing are associated with HIV-1 infection.

TABLE 1
Frequency of CD45 exon A (C77G) mutation in HIV
patients and healthy controls

| | | Number with | |
|---|---|---|---|
| | Total number | Exon A (C77) | Frequency |
| HIV | 197 | 11 | 5.6% |
| Healthy donors | 236 | 4 | 1.7% |

### Example 2

### Abnormal CD45 Splicing in Haemophagocytic Lymphohistiocytosis

Two patients with a similar defect in CD45 splicing associated with familial erythrophagocytic lymphohistiocytosis (Bujan, W., L. Schandene, A. Ferster, C. De Valck, M. Goldman, and E. Sariban. 1993. Lancet 342:1296) and haemophagocytic lymphohistiocytosis (Wagner, R., G. Morgan, and S. Strobel. 1995. Clin. Exp. Immunol. 99:216.) have been previously described. Haemophagocytic lymphohistiocytosis (HLH) is a rare disorder characterised by disregulated activation of T lymphocytes and macrophages (Arico, M., S. Imashuku, R. Clementi, S. Hibi, T. Teramura, C. Danesino, D. A. Haber, and K. E. Nichols. 2001. Blood 97:1131). HLH is genetically heterogenous with both familial and sporadic forms described (Janka, G. E. 1983. Eur. J. Pediatr. 140:221; Dreyer, Z. E., B. L. Dowell, H. Chen, E. Hawkins, and K. L. McClain. 1991. 13:476; Dufourcq-Lagelouse, R., N. Jabado, F. Le Deist, J. L. Stephan, G. Souillet, M. Bruin, E. Vilmer, M. Schneider, G. Janka, A. Fischer, and G. de Saint Basile. 1999. Am. J. Hum. Genet. 64:172).

Because of the similarity of the abnormal CD45 splicing in the two previously described HLH patients, to variant CD45 splicing in apparently normal individuals, we investigated the association of the known C77G mutation and HLH syndrome.

Materials and Methods Materials

Fresh blood was obtained from the previously described family W. (Wagner, R., G. Morgan, and S. Strobel. 1995. Clin. Exp. Immunol. 99:216) and family G. (with two children with HLH) from the Immunobiology Unit, Institute for Child Health, London, UK. PBMC were isolated by centrifugation on a Ficoll-Paque (Amersham Pharmacia Biotech, Buckinghamshire, UK) density gradient and genomic DNA was extracted by standard procedures (Sambrook, J., E. F. Fritsch, and T. Maniatis. 1989. Molecular Cloning: A Laboratory Manual. Cold Spring Harbour Laboratory Press). Genomic DNA samples from family R. (Bujan, W., L. Schandene, A. Ferster, C. De Valck, M. Goldman, and E. Sariban. 1993. Lancet 342:1296.) together with genomic DNA samples from 19 unrelated HLH patients were provided by the Universita di Pavia, Italy.

Genotyping for CD45 Exon A (C77G) Mutation

Genomic DNA was amplified by PCR using forward (5′-GACTACAGCAAAGATGCCCAGTG-3′, SEQ ID NO: 1) and reverse (5′-GGGATACTTGGGTGGAAGTA-3′, SEQ ID NO: 2) primers as previously described (Tchilian, E. Z., D. L. Wallace, N. Imami, Ff. X. Liao, C. Burton, F. Gotcli, J. Martinson, B. F. Haynes, and P. C. Beverley. 2001. J. Immunol. 166:6144.). The C77G transversion introduces a new restriction site for Mspl (Amersham Pharmacia Biotech), which produces two additional fragments of 72 bp and 83 bp upon digestion in the mutant allele. The PCR and digestion products were analysed on VisiGet Separation Matrix (Stratagene, La Jolla, Calif.).

Flow Cytometric Analysis

Flow cytometric analysis of CD45 variant splicing was performed as previously described (Tchilian, E. Z., D. L. Wallace, N. Imami, H. X. Liao, C. Burton, F. Gotch, J. Martinson, B. F. Haynes, and P. C. Beverley. 2001. 166:6144.). Briefly, 2×10⁵ PBMC were stained with APC-conjugated CD3 (Pharmingen, San Diego, Calif.) along with FITC-conjugated CD45RA (Sigma, St Louis, Mo.) and PE-conjugated CD45RO (Dako, Glostrup, Denmark) mAbs in a single step at 4° C. for 20 minutes and washed with PBS, containing 0.5% BSA. Isotype matched mAbs were used as controls. 10,000 events per sample were collected on FACSCalibur (Becton Dickenson, Mountain View, Calif.) and analysed with Cellquest software.

Results CD45 Exon A (C77G) Mutation is the Cause of CD45 Abnormal Splicing in Two Families with HLH

Material was obtained from two patients with HLH, previously described as exhibiting CD45 abnormal splicing as characterised by the lack of the single CD45RO+T cell population (Bujan, W., L. Schandene, A. Ferster, C. De Valck, M. Goldman, and E. Sariban. 1993. Lancet 342:1296; Wagner, R., G. Morgan, and S. Strobel. 1995. Clin. Exp. Immunol. 99:216). Subsequently a C77G mutation in exon A of CD45 has been shown to be responsible for the abnormal CD45 splicing in T lymphocytes (Thude, H., J. Hundrieser, K. Wonigeit, and R. Schwinzer. 1995. Eur. J. Immunol. 25:2101; Zilch, C. F., A. M. Walker, M. Timon, L. K. Goff, D. L. Wallace, and P. C. Beverley. 1998. Eur. J. Immunol. 28:22). We therefore genotyped these patients and members of their families for the presence of the CD45 exon A C77G mutation.

Patient W. was the third child of healthy unrelated British Caucasian parents (Wagner, R., G. Morgan, and S. Strobel. 1995. Clin. Exp. Immunol. 99:216.). He presented aged 3 mo with fever, diarrhoea, pallor, increasing irritability and marked cervical lymphoadenopathy and hepatosplenomegaly. Laboratory investigations revealed pancytopenia, coagulopathy and hypertryglyceridemia. The diagnosis of HLH was made from the bone marrow aspirate, which showed haemophagocytosis. There was a good response to initial treatment with dexametliasone and etoposide and he underwent allogeneic bone marrow transplantation from his HLA identical brother.

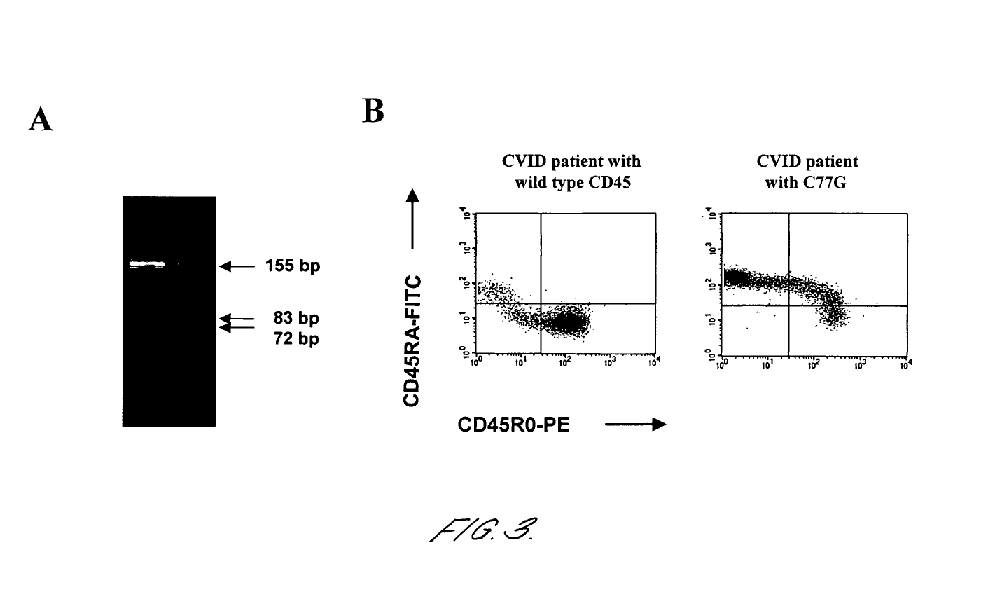

Using PCR and MspI restriction analysis we found that patient W. his mother and two siblings were heterozygous for the mutant C77G allele while the father and the oldest brother had wild type CD45 (FIG. 2A). These results were confirmed by flow cytometric analysis on PBMC from family W. (FIG. 2B). All of the family members genotyped as having the C77G mutation exhibit phenotypically abnormal CD45 splicing. (These results are in agreement with the initial report of family W. that at that time had only three children.)

Patient R. was a first child of consanguineous Belgian Caucasian parents (Bujan, W., L. Schandene, A. Ferster, C. De Valck, M. Goldman, and E. Sariban. 1993. Lancet 342:1296.). The patient presented at the age of 2 mo with fever, hepatosplenomegaly, neutropenia, thrombocytopenia, hypofibrinogenemia and hypertriglyceridemia. He responded to initial treatment with etoposide and underwent bone marrow transplantation from his haploidentical half-sibling and remained asymptomatic over 8 years later. Two older siblings from a previous marriage died during infancy of a histiocytic disorder. Genotyping for the C77G polymorphism revealed that the patient and his mother are heterozygotes while his father and grandmother (also the father's sister) carried wild type CD45 (data not shown). Taken together these results show that the CD45 exon A (C77G) mutation is the cause for the CD45 abnormal splicing in the two HLH patients.

Analysis of CD45 Exon A C77G Mutation in 21 HLH Patients

Since two families with HLH were identified with abnormal CD45 splicing and the C77G mutation we next investigated the pattern of CD45 expression in other HLH patients. Using PCR and MspI restriction analysis we genotyped 21 patients with HLH (including the two affected sibs from family G.) for the presence of CD45 exon A (C77G) mutation. We did not find the mutant C77G allele in any of these patients.

Although taken together the above results show a frequency of 1:10 in HLH type 2 (with identified mutations in the PRFI gene), or 2:23 for HLH overall, the number of subjects included in the study was very small and it is therefore impossible to draw statistically significant conclusions. Extensive studies on the frequency of C77G have not been performed but we have shown the frequency of the C77G individuals to be about 1.76% in the UK (Tchilian, E. Z., D. L. Wallace, N. Imami, 14. X. Liao, C. Burton, F. Gotch, I. Martinson, B. 1F. Haynes, and P. C. Beverley. 2001. J. Immunol. 166:6144.), while in Germany the frequency has been reported to be less then 1% and in North America to be higher (3.6 %) (Jacobsen, M., D. Schweer, A. Ziegler, R. Gaber, S. Schock, R. Schwinzer, K. Wonigeit, R. B. Lindert, 0. Kantarci, J. Schaefer-Klein, H. I. Schipper, W. H. Oertel, F. Heidenreich, B. G. Weinshenker, N. Sommer, and B. Hemmer. 2000. Nat. Genet. 26:495.).

### Example 3

### Abnormal CD45 Splicing in a Patient with a Common Variable Immunodeficiency and a History of Prolonged Faecal Excretion of Poliovirus.

Common variable immunodeficiency (CVID) is an acquired primary antibody deficiency characterised by recurrent encapsulated bacterial infection and autoimmune disease. The underlying pathogenic defects are heterogeneous with at least four groups of patients being identified according to their ability to secrete immunoglobulin in vitro [Bryant A, Calver N.C., Toubi E, Webster AD, Farrant J. Clin Immunol Immunopathol 1990; 56: 239-48], presence of granulomatous disease and autoimmune disease. In general, patients with CVID are not prone to viral infections though infection with enteroviruses may be a potential problem [Rudge P, Webster AD, Revesz T, et al. Brain 1996; 119: 1-15.]. In view of the possibility that abnormalities in CD45 splicing might contribute to impaired anti-viral responses we report here on a patient with CVID and a history of prolonged poliovirus excretion [Misbah SA, Lawrence Penn., Kurtz JB, Chapet HM. Postgrad Med J 1991; 67: 301-303], who exhibited abnormal CD45 splicing caused by the C77G polymorphism.

Materials and Methods Case History

The patient was a 49 year old Caucasian male with CVID who had prolonged faecal excretion of a non-vaccine strain type II poliovirus between January 1987 and July 1988. In view of his occupation as a nurse and the attendant occupational health implications of prolonged poliovirus excretion, his case history was previously reported [Misbah SA, Lawrence Penn., Kurtz JB, Chapet HM. Postgrad Med J 1991; 67: 301-303.]. In brief CVID was diagnosed at the age of 18 years when he presented with hypogammaglobulinaemia [IgG2.8g/l (ref. range 8-16), IgA 0.48 g/l (ref. range 1.4-4.2), IgM undetectable (ref. range 0.5-2.0)] on a background of delayed puberty, intermittent diarrhoea and intestinal nodular lymphoid hyperplasia. H-e was lost to follow-up between 1972 and 1986. Although he did not suffer from recurrent infections, it was thought prudent to commence him on intramuscular immunoglobulin therapy in January 1987 because of his occupation as a nurse. He has been maintained on replacement immunoglobulin since, switching from intramuscular to subcutaneous immunoglobulin in September 1998. Trough serum IgG levels have varied between 4.4 to 6.1 g/l over the past 2 years. His clinical progress on immunoglobulin replacement has been excellent with only occasional episodes of diarrhoea.

Materials

Fresh EDTA blood was obtained from the patient [Misbah SA, Lawrence PA, Kurtz JB, Chapel HM. Postgrad Med J 1991; 67: 301-303.] via the Department of Immunology, John Radcliffe Hospital, Oxford, UK. Genomic DNA was extracted by standard procedure [Sambrook J, E. F. Fritsch, and T. Maniatis. Molecular Cloning: A Laboratory Manual. Cold Spring Harbour Laboratory Press 1989] and monoclonal antibody staining performed as described below.

Genotyping for CD45 Exon A (C77G) Mutation

Genomic DNA was amplified by PCR using forward (5′-GACTACAGCAAAGATGCCCAGTG-3′, SEQ ID NO: 1) and reverse (5′-GGGATACTTGGGTGGAAGTA-3′, SEQ ID NO: 2) primers as previously described [Tchilian EZ, Wallace DL, Imami N, et al. J Immunol 2001; 166: 6144-8]. The C77G transversion introduces a new restriction site for Mspl (Amersham Pharmacia Biotech), which produces two additional fragments of 72 bp and 83 bp upon digestion in the mutant allele. The PCR and digestion products were analysed on a VisiGet Separation Matrix (Stratagene, La Jolla, Calif.).

Flow Cytometric Analysis

Flow cytometric analysis of CD45 variant splicing was adapted from the method previously described [Tchilian EZ, Wallace DL, Imami N, et al. J Immunol 2001; 166: 6144-8.]. Briefly, 50 μl of EDTA blood was stained with PerCP-conjugated CD3, FITC-conjugated CD45RA and PE-conjugated CD45RO (Becton Dickinson, Oxford, UK) monoclonal antibodies for 15 minutes in the dark at room temperature. Red blood cells were lysed by addition of 1 ml of FACSlyse (Becton Dickinson) for 10 minutes. Lysed stained cells were washed twice with sheath fluid (Becton Dickinson) before being fixed in 0.4ml of 1% paraformaldehyde and analysed on a FACScan flow cytometer (Becton Dickinson) using Cellquest software. 10,000 events per sample were collected and isotype matched mAbs were used as controls.

Results

Using PCR and MspI restriction analysis we found that the patient was heterozygous for the mutant C77G allele (FIG. 3A). This result was confirmed by flow cytometric analysis on PBMC froth the patient. As shown on FIG. 3B the variant pattern of CD45 splicing can be identified by the absence of the single CD45RO+T cell population. Taken together these results show that the patient exhibits abnormal CD45 splicing caused by the C77G polymorphism in the gene encoding CD45.

GenBank Accession Numbers

M23461 (SEQ ID NO:3) human PTPRC gene exons 1 and 2

M23462 (SEQ ID NO:4) human PTPRC gene exon 3

M23494 (SEQ ID NO:5) human PTPRC gene exon 4

M23495 (SEQ ID NO:6) human PTPRC gene exon 5

M23496 (SEQ ID NO:7) human PTPRC gene exon 6

M23466 (SEQ ID NO:8) human PTPRC gene exon 7

M23467 (SEQ ID NO:9) human PTPRC gene exon 8

M23468 (SEQ ID NO: 10) human PTPRC gene exon 9

M23469 (SEQ ID NO: I 1) human PTPRC gene exon 10

M23470 (SEQ ID NO: 12) human PTPRC gene exon 11

M23471 (SEQ ID NO:13) human PTPRC gene exon 12

M23472 (SEQ ID NO:14) human PTPRC gene exon 13

M23473 (SEQ ID NO: 15) human PTPRC gene exon 14

M23474 (SEQ ID NO: 16) human PTPRC gene exon 15

M23475 (SEQ ID NO: 17) human PTPRC gene exon 16

M23476 (SEQ ID NO: 18) human PTPRC gene exon 17

M23477 (SEQ ID NO:19) human PTPRC gene exon 18

M23478 (SEQ ID NO:20) human PTPRC gene exon 19

M23479 (SEQ ID NO:21) human PTPRC gene exon 20

M23480 (SEQ ID NO:22) human PTl PRC gene exon 21

M23481 (SEQ ID NO:23) human PTPRC gene exon 22

M23482 (SEQ ID NO:24) human PTPRC gene exon 23

M23483 (SEQ ID NO:25) human PTPRC gene exon 24

M23484 (SEQ ID NO:26) human PTPRC gene exon 25

M23485 (SEQ ID NO:27) human PTPRC gene exon 26

M23486 (SEQ ID NO:28) human PTPRC gene exon 27

M23487 (SEQ ID NO:29) human PTPRC gene exon 28

M23488 (SEQ ID NO:30) human PTPRC gene exon 29

M23489 (SEQ ID NO:31) human PTPRC gene exon 30

M23490 (SEQ ID NO:32) human PTPRC gene exon 31

M23491 (SEQ ID NO:33) human PTPRC gene exon 32

M23492 (SEQ ID NO:34) human PTPRC gene exon 33

1. A method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

2. A method according to claim 1 which comprises screening for the presence or absence in the human subject of the C77G mutation in the gene encoding CD45, wherein subjects having at least one mutant allele are scored as being more susceptible to viral infection and/or more pre-disposed to developing severe disease following viral infection, as compared to subjects who do not carry a C77G mutation.

3. A method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression associated with the presence of a C77G mutant allele of the gene encoding CD45 is taken as an indication that the subject is more susceptible to viral infection and/or more pre-disposed to developing severe disease following viral infection, as compared to subjects who do not carry a C77G mutation.

4. A method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression associated with the presence of a C77G mutant allele of the gene encoding CD45 is taken as an indication that the subject is more susceptible to viral infection and/or more pre-disposed to developing severe disease following viral infection, as compared to subjects who do not carry a C77G mutation.

5. A method according to any one of claim 1 wherein the viral infection is infection with a human immunodeficiency virus.

6. A method according to claim 5 wherein the human immunodeficiency virus is HIV-1.

7. A method according to any one of claim 1 wherein the viral infection is infection with EBV.

8. A method according to any one of claim 1 wherein the viral infection is infection with poliovirus.

9. A method of screening a human subject for susceptibility to developing immunodeficiency disease other than severe combined immune deficiency and/or pre-disposition to a developing a more severe form of immunodeficiency disease, which method comprises screening for the presence or absence in the genome of said subject of one or more polymorphic variants or mutations in the gene encoding CD45 or of one or more polymorphic variants in linkage disequilibrium with or in close physical proximity to a polymorphic locus in the gene encoding CD45.

10. A method as claimed in claim 9 which comprises screening for the presence or absence in the human subject of the C77G mutation in the gene encoding CD45, wherein subjects having at least one mutant allele are scored as being more susceptible to developing immunodeficiency and/or more likely to develop a severe form of immunodeficiency than subjects who do not carry a C77G mutation.

11. A method of screening a human subject for susceptibility to developing immunodeficiency disease other than severe combined immune deficiency and/or pre-disposition to a developing a more severe form of immunodeficiency disease, which method comprises evaluating the pattern of CD45 mRNA expression in the subject, wherein the presence of an abnormal pattern of CD45 mRNA expression associated with the presence of a C77G mutant allele of the gene encoding CD45 is taken as an indication that the subject is more susceptible to developing immunodeficiency and/or more likely to develop a severe form of immunodeficiency than subjects who do not carry a C77G mutation.

12. A method of screening a human subject for susceptibility to developing immunodeficiency disease other than severe combined immune deficiency and/or pre-disposition to a developing a more severe form of immunodeficiency disease, which method comprises evaluating the pattern of CD45 protein expression in the subject, wherein the presence of an abnormal pattern of CD45 protein expression associated with the presence of a C77G mutant allele of the gene encoding CD45 is taken as an indication that the subject more susceptible to developing immunodeficiency and/or more likely to develop a severe form of immunodeficiency than subjects who do not carry a C77G mutation.

13. A method of screening a human subject for susceptibility to haemophagocytic lymphohistiocytosis, which method comprises screening for the presence or absence in the human subject of the C77G mutation in the gene encoding CD45, wherein subjects having at least one mutant allele are scored as being more susceptible to haemophagocytic lymphohistiocytosis, as compared to subjects who do not carry a C77G mutation.

14. A method of investigating the genetic basis of the disease haemophagocytic lymphohistiocytosis in a human subject previously diagnosed with haemophagocytic lymphohistiocytosis, which method comprises screening for the presence or absence in the human subject of the C77G mutation in the gene encoding CD45, wherein the presence of at least one mutant allele is taken as an indication that the mutation may contribute to the genetic basis of haemophagocytic lymphohistiocytosis in the subject.
